(19) United States
(12) Patent Application Publication
(10) Pub. No.: US 2024/0312601 A1
(43) Pub. Date: Sep. 19, 2024

(54) SYSTEM AND METHOD FOR INDIVIDUALIZED HEALTHCARE RECOMMENDATIONS

(71) Applicant: Barnard Health Research Limited, Fareham (GB)

(72) Inventor: Katharine Denise Kelly, Fareham (GB)

(21) Appl. No.: 18/671,396

(22) Filed: May 22, 2024

Related U.S. Application Data
(63) Continuation of application No. 17/308,829, filed on May 5, 2021.
(60) Provisional application No. 63/153,798, filed on Feb. 25, 2021.

(30) Foreign Application Priority Data
May 9, 2020 (GB) 2006864.9

(51) Int. Cl. G16H 20/70; G16H 10/60; G16H 40/67; G16H 50/20

(52) U.S. Cl. CPC G16H 20/70; G16H 10/60; G16H 40/67; G16H 50/20

(57) ABSTRACT

A computer-implemented method of formulating a holistic treatment recommendation. The method includes receiving a first set of patient data based on responses to a first set of prompts relating to a plurality of priority concern areas associated with self-care management and determining, using a first algorithm, which of the priority concern areas are relevant to the patient. The method further includes receiving a second set of patient data based on responses to a second set of prompts relating to the one or more of the priority concern areas determined to be relevant to the patient and determining, using a second algorithm, which of the priority concern areas is most relevant to the patient. The method further includes synthesizing and analyzing the second set of patient data and the patient's electronic medical health data to determine a holistic treatment recommendation for the patient, which is then output on a display.

### CROSS REFERENCE TO RELATED APPLICATIONS

This application is a continuation of pending U.S. Non-Provisional application Ser. No. 17/308,829, filed May 5, 2021, which claims priority to United Kingdom Patent Application No. 2006864.9, filed May 9, 2020, and U.S. Provisional Patent Application No. 63/153,798, filed Feb. 25, 2021, the contents of each of which are incorporated herein by reference in their entireties.

### FIELD

The embodiments of the present invention relate to systems and methods of improving patient care. Specifically, the embodiments described herein relate to identification of personal barriers to optimal self-care behaviors, taking into account factors such as, e.g., a patient's knowledge, social support, psychological burden, and therapy aspects and providing a holistic treatment recommendation in response thereto.

### BACKGROUND

Presently, routine patient care visits leave people with chronic health conditions or diseases (e.g., diabetes (pre-diabetes, type 1, or type 2), irritable bowel disease, chronic obstructive pulmonary disorder, cancer, cardiovascular disease, mental health, chronic pain, and the like) and their healthcare professionals (HCPs), both in primary and specialist care settings, feeling frustrated. Presently, healthcare visits are typically designed to focus on biomedical outcomes of conditions by using a didactic medical model. Regimens for dealing with chronic conditions are often complex, requiring multi-factorial risk reduction strategies. These may include ongoing self-management, education, support, and the like to maintain optimal control and prevent long-term complications, which are currently not well accounted for.

Much of the responsibility for self-management lies with the person with a chronic condition, but support from his/her healthcare professionals is essential. Although it is assumed that the vast majority of healthcare providers are dedicated to providing the highest quality of care, healthcare systems often struggle to create organizational and structural changes necessary to offer support for factors that are not disease-specific. Possible contributing factors include the HCP's lack of time, burnout, lack of sufficient patient disease-specific and/or lifestyle-specific data, inadequate knowledge, or the like. Moreover, many patient needs, concerns, and barriers are not discussed during healthcare visits, and patients and physicians often disagree on the central problem presented. Disagreement about treatment goals, inconsistency among healthcare teams, and confusion about treatment priorities are associated with poorer outcomes. In addition, goals mutually agreed-upon by the patient and his/her HCP are often not followed up on, leaving patients frustrated and HCPs struggling to provide tailored support.

Facilitating improved interaction between HCPs and patients by exploring informed, co-decision making opportunities and providing possible care pathways would enhance patient and HCP experiences, empower patients, improve decision-making, and personalize healthcare—ultimately achieving improved biomedical, psychosocial, and quality of life outcomes.

Such a paradigm shift away from a purely medical model to one with a greater emphasis on psychosocial aspects associated with chronic conditions fostering a more holistic, person-centered approach resulting in more effective self-management has long been advocated but not successfully implemented. There is a widespread lack of understanding of the impact of patient-specific psychosocial burden associated with his/her condition, the evolving consequences, and how to address them. Specifically, the impact of insufficient knowledge, social factors, psychological burden, and therapy aspects, which can affect a patient's ability/willingness to follow a treatment regime, are often underrated or not considered at all. This often results in a negative impact on clinical practice with consequential negative outcomes for patients and increasing frustration for and with HCPs.

There is an urgent need to translate existing components of care into individualized, practical, collaborative, and enhanced chronic condition management that is respectful of and responsive to individual patient preferences, needs, and values, with the patient at the heart of decision-making and supported by his/her HCP. Currently, there exist at least two fundamental challenges to doing so: (1) patients are often unable to understand/articulate their priorities, facilitators, and/or barriers to self-management behaviors; and (2) HCPs are often unable to understand the broader psychosocial context of disease management beyond the medical model of healthcare. This results in frustration for both parties, a deterioration in therapeutic relationships, and poor outcomes for both parties.

The proliferation of technologies for the treatment of chronic conditions is undeniable. However, technology alone is insufficient to achieve optimal outcomes and, in fact, can sometimes contribute to greater feelings of despair and distress. For example, existing mobile applications, websites, and integrated care platforms often do not address the core issue affecting poor outcomes for many people dealing with chronic conditions or the burgeoning burden on HCPs supporting them. Instead, a holistic, individualized and psychosocially empathic approach targeting quality of life outcomes is needed. Such an intervention can lead to long-term improvements and efficiencies in the patient/professional interface in routine clinical outpatient appointments, thereby improving patient care and HCP experiences and leading to improved outcomes for people with chronic conditions.

It is an object of the embodiments disclosed herein to provide a novel care platform for HCPs and patients to facilitate collection of relevant information for the identification and agreement of best possible pathways for improved patient outcomes. This information may be used to provide an improved system for understanding the patient's personal barriers and for providing a range of available healthcare options and care pathways (within the context of, e.g., medical, social and environmental aspects). The results may be provided directly to the patient, his/her HCP, and/or healthcare system and can be tailored to that individual patient in a collaborative, co-goal-setting environment with the HCP, ultimately leading to long-term improvements of quality of life outcomes for people with chronic conditions.

### SUMMARY

According to aspects disclosed herein, a computer-implemented method of formulating a holistic treatment recommendation for individualized care is disclosed. The method is performed by one or more processors and comprises receiving a first set of patient data from a patient database stored in a memory device. The first set of patient data is based on responses to a first set of prompts relating to a plurality of priority concern areas associated with self-care management. The method further includes determining, using a first algorithm giving predetermined weights to each of the first set of prompts or responses thereto, which of the priority concern areas are relevant to the patient. The method further comprises receiving a second set of patient data from the patient database stored in the memory device. The second set of patient data is based on responses to a second set of prompts relating to the one or more of the priority concern areas determined to be relevant to the patient. The method further comprises determining, using a second algorithm giving predetermined weights to each of the second set of prompts or responses thereto, which of the priority concern areas is most relevant to the patient. The method further comprises retrieving the patient's electronic medical health data and synthesizing and analyzing the second set of patient data and the electronic medical health data to determine a holistic treatment recommendation for the patient. The method further comprises outputting, on a display associated with the memory device, the holistic treatment recommendation for the patient.

According to further aspects disclosed herein, a computing system is disclosed. The computing system includes one or more computers and one or more storage devices storing instructions that are operable, when executed by the one or more computers, to cause the one or more computers to perform operations. The operations include receiving a first set of patient data from a patient database stored in a memory device. The first set of patient data is based on responses to a first set of prompts relating to a plurality of priority concern areas associated with self-care management. The operations further include determining, using a first algorithm giving predetermined weights to each of the first set of prompts or responses thereto, which of priority concern areas are relevant to the patient. The operations further include receiving a second set of patient data from the patient database stored in the memory device. The second set of patient data is based on responses to a second set of prompts relating to the one or more of the priority concern areas determined to be relevant to the patient. The operations further include determining, using a second algorithm giving predetermined weights to each of the second set of prompts or responses thereto, which of the priority concern areas is most relevant to the patient. The operations further include retrieving the patient's electronic medical health data and synthesizing and analyzing the second set of patient data and the electronic medical health data to determine a holistic treatment recommendation for the patient. The operations further include outputting, on a display associated with the memory device, the holistic treatment recommendation for the patient.

It is to be understood that both the foregoing general description and the following detailed description are exemplary and explanatory only and are not restrictive of the invention as claimed.

### BRIEF DESCRIPTION OF THE DRAWINGS

These and other features, aspects, and advantages of the present invention will become apparent from the following description, appended claims, and the accompanying exemplary embodiments shown in the drawings, which are briefly described below.

FIG. 1 is a flowchart illustrating a method of formulating a holistic treatment recommendation for individualized care according to one embodiment.

FIG. 2A shows an exemplary patient home page, according to one embodiment.

FIG. 2B shows an exemplary patient home page, according to another embodiment.

FIG. 3 shows an exemplary healthcare professional home page, according to one embodiment.

FIG. 4 illustrates an exemplary resource page for type 1 diabetes, according to one embodiment.

While the invention is susceptible to various modifications and alternative forms, specific forms thereof have been shown by way of example in the drawings and will herein be described in detail. It should be understood, however, that it is not intended to limit the invention to the particular forms disclosed, but, on the contrary, the intention is to cover all modifications, equivalents, and alternatives falling within the spirit and scope of the invention.

### DETAILED DESCRIPTION

Each of the above embodiments and obvious variations thereof are contemplated as falling within the spirit and scope of the claimed invention, which is set forth in the following claims. Moreover, the present concepts expressly include any and all combinations and sub-combinations of the preceding elements and aspects.

The systems and methods described herein provide effective e-tools in routine primary and secondary care settings for individuals with chronic health conditions, illnesses, or diseases (e.g., pre-diabetes, type 1 diabetes (T1D), type 2 diabetes (T2D), irritable bowel disease, chronic obstructive pulmonary disorder, cancer, cardiovascular disease, mental health, chronic pain, and the like).

The systems and methods described herein may be used to identify the main personal facilitators and barriers to an individual effectively self-managing his/her chronic health condition, providing personalized healthcare for the individual according to his/her current situation, delivered with HCP support. The systems and methods may be tailored to individual needs when implemented in clinical practice. The systems and methods are designed specifically for routine clinical practice to reduce the burden on HCPs, directly facilitating national roll-out at low cost, with little or no increase in outpatient visit time. The systems and methods may be used to reduce the public health burden of chronic health conditions and associated long-term complications.

Consultations implementing the systems and methods described herein aim to reduce the burden on healthcare professionals by removing the pressure to know all of the information required for best-practice decision-making, especially when patients often are unwilling or unable to articulate the required information. Interactions between individuals and healthcare professionals using the tools and methods described herein are enhanced by identifying the specific obstacles, goals, and questions important to an individual, providing choices of care pathways available to them (via a computer modelling algorithm), and providing resources for those pathways, thereby helping HCPs provide tailored healthcare in a collaborative, joint goal-setting, co-decision-making approach to maximize appointment time and specifically address the individual's concerns. The systems are monitored (securely with strict confidentiality and unique user settings), in real-time, durable, and tailored.

The system according to the embodiments disclosed herein incorporates addresses primarily the clinical aspects of care and self-management (e.g., medications, devices, treatment regimens, lifestyle, and education), and pulls these together into a holistic wide-reaching tool that includes both external factors (social support, available resources, therapy aspects, and environment) and internal factors (knowledge, intrinsic feelings, beliefs, and motivations) to address a full range of aspects of the patient lived experience. Self-management behavior is influenced by each aspect to some extent at any given time. The unique component parts, while individually distinct, are linked to interact and reflect the patient's experience and priorities for his/her specific needs and, as such, drive understanding and subsequent actions.

Personalized healthcare is provided by showing the individual's priorities, alongside clinical outcomes in a way that aids explanation of available individualized specific care pathways to increase individual awareness and self-efficacy, enhance self-management, empower greater management and control of the patient's own health, improve metabolic control, reduce acute and long-term complications, have a positive impact on quality of life and psychosocial functioning, reduce non-adherence to prescribed medication, create successful outcomes including enhanced quality of life and psychosocial functioning, and the like. Thus, self-management is situated more effectively in the context of each person's lifestyle and personal priorities. In addition, social inequalities in terms of access to healthcare should be recognized and addressed through a broader understanding of—and, ideally, the removal of—unconscious, inherent HCP bias. This approach differs from traditional interventions by repositioning control of chronic condition management to optimize patient empowerment and awareness, reduce personal uncertainties, explore whole-life factors, and improve interactions between individuals and his/her HCP.

The embodiments described herein disclose a technological system and tool underpinned by evidence-based, theory-driven psychological behavioral decision-making algorithms. In essence, the system comprises a pre-clinic assessment, including a brief, algorithmic, “smart,” adaptive, survey-based questionnaire completed on a computing device (e.g., a smart phone, tablet, computer, other device with a web connection, or the like) by an individual having a chronic condition. The questions or prompts in the assessment are generally related to a patient's self-management concerns, incorporating biomedical, psychological burden, social support, and therapy factors to determine optimal treatment pathway(s). The assessment is designed to improve consultations by rapidly identifying patient priorities determined using an algorithm.

Patient priorities determined from the brief, core assessment are then linked with external sources including to relevant data, such as electronic medical records, diagnostic and medical tests/examinations, personal health and fitness trackers, sleep monitors, and/or other device(s) and with personal computing devices to determine optimal treatment pathways. The system then processes and organizes this information, based on user priorities, and presents the identified patient priorities and optimal pathways to aid consultations in routine care. The information may be presented in various ways including graphically, figuratively, written text, audio, and/or color presentation. The core component may utilize a decision-tree based format such that it is easily read and interpreted.

The component parts aiding the formulation of the treatment recommendation for individualized care include external factors, internal regimen factors, and personal factors affecting behavior that are used to form the basis for an individualized care plan. The proposed care plan may be checked and revised by the patient and/or his/her HCP and revised, if needed, to arrive at an acceptable and sustainable individualized care plan. More specifically, and as discussed in detail below, as individual priorities for each component part are assessed and prioritized, available care pathways are mapped by unique algorithms to each priority to provide choice and become the focus of clinical discussion. In one non-limiting example, “social support,” “education,” and “beliefs” may take priority at one time; however, this may change to, e.g., “environment,” “feelings,” and “education,” as life circumstances change. Thus, the level of social support and education that an individual requires on diagnosis may subside as motivation and feelings take priority over time. Changes to therapy or choices of devices may bring education back to the fore and highlight resource needs and health beliefs.

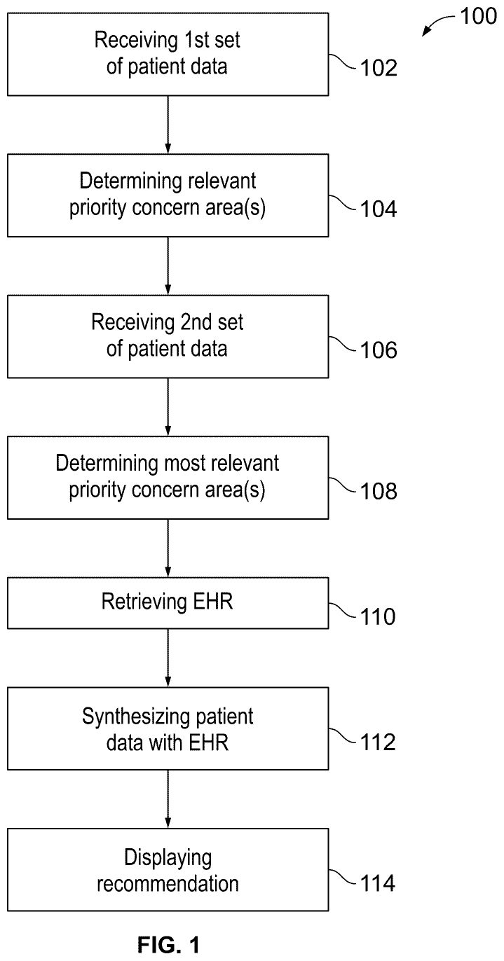

Referring to FIG. 1, a non-limiting example of a computer-implemented method 100 of formulating a holistic treatment recommendation for individualized care is shown. The method is performed by one or more processors. The method includes receiving a first set of patient data from a patient database stored in a memory device at step 102. The first set of patient data may be based on responses to a first set of prompts relating to a plurality of priority concern areas associated with self-care management. The method further includes determining, using a first algorithm giving predetermined weights to each of the first set of prompts or responses thereto, which of the priority concern areas are relevant to the patient at step 104. At step 106, a second set of patient data is received from the patient database stored in the memory device. The second set of patient data may be based on responses to a second set of prompts. The second set of prompts may relate to the one or more of the priority concern areas determined to be relevant to the patient. At step 108, a second algorithm giving predetermined weights to each of the second set of prompts or responses thereto is used to determine which of the priority concern areas is most relevant to the patient. The patient's electronic medical health data is retrieved at step 110. As step 112, the method includes synthesizing and analyzing the second set of patient data and the electronic medical health data to determine a holistic treatment recommendation for the patient. At step 114, the holistic treatment recommendation for the patient is output on a display associated with the memory device.

The outputs of the systems and methods discussed herein can be discussed in routine consultations with HCPs to focus treatment (or other) decisions tailored to the unique needs of the patient to improve adherence, engagement, and ownership of those decisions. Intrinsic motivation (e.g., decisions that patients “own”) is known to be considerably more effective than externally imposed instructions. A key strength of the disclosed system is that it brings all relevant information together in one place in an easily accessible way to support optimal outcomes for users. Thus, the system enables enhanced consultations during routine care providing agreement based on informed choice of appropriate care pathways, leading to optimal outcomes for both patients and HCPs.

The system and method of the embodiments described herein will now be discussed in more detail according to non-limiting embodiments.

When a patient having a chronic health condition schedules a routine outpatient appointment or consultation, the clinic or HCP may send a link to a pre-clinic assessment (e.g., a survey or questionnaire) to the patient that is assessable via the patient's computing device (e.g., smartphone, tablet, computer, or other device having a web connection). The patient may log in to his/her account and complete the assessment prior to his/her appointment.

Prior to or included within the assessment, prompts may be displayed on the patient's computing device asking the patient to enter preliminary information. Such preliminary information may include, but is not limited to, gender, age, duration of condition, type of therapy, frequency of monitoring condition, treatment, any combination thereof, or the like. The patient may also be asked to specify his/her areas of concerns and/or topics that he/she would like to discuss during the appointment.

The prompts included in the assessment may be unique to the patient based on his/her condition, previous assessment results, or the like. In some embodiments, the assessment includes quick and “easy-to-answer” prompts or questions. For example, it may take a user from about 1 to about 10 seconds to answer each prompt.

The prompts may be questions or statements (e.g., for which the patient indicates agreement or disagreement). In some embodiments, the patient may respond to the statements by selecting “yes” or “no,” “true” or “false,” indicating how/strongly he/she feels about a certain statement/topic, any combination thereof, or the like. In some embodiments, the patient may respond by ranking his/her level of agreement, e.g., using a numerical scale, e.g., 1-5, where 1 indicates strong disagreement with the prompt, 5 indicating strong agreement with the prompt), and so on. In some embodiments, the patient may choose between the following response options: agree totally, agree somewhat, disagree somewhat, and disagree totally. The subject matter to which the prompt relates and/or number of prompts included in the assessment may be determined in real-time, depending on how the patient answers preceding prompts and which areas of concern he/she identifies as a priority. In some non-limiting embodiments, the predetermined amount of prompts is about 10-60. In some non-limiting embodiments, the predetermined amount of prompts is about 20-50. In some non-limiting embodiments, the predetermined amount of prompts is about 25-45. The prompts are generally designed to address holistic aspects of living with a chronic condition. Thus, they may be related to priority concern areas relating to self-management skills, including knowledge, social support, psychological burden, and therapy aspects.

The prompts and/or responses thereto are weighted within themselves and/or against other relevant prompts to identify the patient's key priority concerns. Based on the patient's responses and the weight given to each of the prompts/responses, the patient is guided through tailored and specific prompts relevant to that patient. The weighted prompts/responses are dynamically calculated during the assessment so that the patient is later presented only with the top priority concern areas that best reflect his/her responses. In some embodiments, greater weight is given to responses indicating an area of concern for the patient. In some embodiments, greater weight is given to areas determined to be more serious. For example, an indication that the patient feels depressed due to his/her condition may be more heavily weighted than an indication that the patient feels burdened by his/her condition. In some embodiments, the weight given to a strong agreement or strong disagreement with the prompt is greater than the weight given for mild agreement or mild disagreement with the prompt.

In one non-limiting example, the patient's previous responses may be used to narrow the subject matter of the prompts. For example, based on the patient's previous responses, he/she may be directed to further questions on only two the original four priority concern areas, e.g., psychological burden and social support, psychological burden and therapy aspects, psychological burden and knowledge, social support and knowledge, social support and therapy aspect, or therapy aspects and knowledge.

In some embodiments, the assessment includes a first and second set or “wave” of prompts. A first set of patient data, based on responses to the first wave of prompts tailored to the patient's particular condition, may be received by the computing device from patient input and stored in a memory device. The first set of patient data may be based on responses to a first set of prompts relating to a plurality of aspects of priority concern areas relating to self-care management. The prompts in the first wave may include higher-level, broader questions. Examples of priority concern areas may include knowledge, social support, psychological burden, therapy aspects, any combination thereof, and the like.

First wave prompts relating to knowledge may, more specifically, relate to understanding the patient's treatment regiment, use of medical devices, understanding medication dosages, understanding how lifestyle (e.g., diet, exercise) choices can affect outcomes, self-management skills required for optimal healthcare outcomes, accurately recalling previous test results, any combination thereof, or the like. First wave prompts relating to social support may, more specifically, relate to availability of support resources, support from family and friends, support from HCPs, whether the patient's environment is conducive to managing his/her condition, a combination thereof, or the like. First wave prompts relating to psychological burden may, more specifically, relate to ability to cope with demands of the condition, feeling sad, scared, and/or overwhelmed about the condition, feeling distressed about the condition, feeling depressed or hopeless about the condition, feeling over-burdened or bothered by the condition, ability to adhere to and remember to implement treatment plans, motivation to self-manage the condition, any combination thereof, or the like. First wave prompts relating to therapy may, more specifically, relate to concerns about oral medications and/or injectable therapies, lifestyle concerns relating to the condition, concerns about effectively managing the condition, feeling over-burdened or hassled by treatment, feeling that current treatment is ineffective, accessibility and/or use of devices that assist with treating the condition, complications related to the condition, side effects of the patient's medication, comfort with education level relating to the condition, any combination thereof, or the like.

A first algorithm is then used to give predetermined weights to each of the first set of prompts or responses thereto to determine which of the priority concern areas is/are most relevant to the patient. A second set or wave of prompts tailored to the priority concerns areas determined to be the most relevant to the patient may then be provided to the patient. Thus, prompts relating to the priority concern areas not determined to be particularly relevant to the patient may be eliminated from the second wave of prompts.

A second set of patient data, based on responses to the second wave of prompts tailored to the patient's particular condition, may be received by the computing device from patient input and stored in a memory device. The second set of patient data is based on responses to the second set of prompts associated with one or more of the priority concern areas determined using the first algorithm applied to the answers to the first wave of prompts. In some embodiments, the second set of prompts includes questions/statements that are more narrowly tailored or specific to the priority concern areas determined to be the most relevant to the patient or that relate to care pathways associated with the priority concern areas determined to be the most relevant to the patient.

Second wave prompts relating to knowledge may, more specifically, relate to confidence in understanding how the patient's condition is affected by exercise and/or diet, knowing how to respond to various test results, confidence in adjusting medication when needed, confidence in use of medical devices relating to the condition, any combination thereof, or the like. Second wave prompts relating to social support may, more specifically, relate to level of help relating to condition from friends and family, feeling of emotional support relating to condition from friends and family, feeling that friends and family worry too much about the condition, feeling that work colleagues understand the demands of the condition and give appropriate space needed to manage it, concerns about loss of independence, any combination thereof, or the like. Second wave prompts relating to psychological burden may, more specifically, relate to frequency of worry about condition-related complications, feeling overwhelmed when thinking about living with the condition, feeling sad when thinking about living with the condition, feeling scared, anxious, depressed, or angry when thinking about living with the condition, feeling like steps taken to manage condition are never enough, confidence that help can be sought when needed, fear of future complications, impact of condition on future quality of life, any combination thereof, or the like. Second wave prompts relating to therapy aspects may, more specifically, relate to effectiveness of current treatment, confidence in managing sick days, whether therapy is helping to manage pregnancy-related issues, feeling that current therapy requires too much time, is too complicated, or limits spontaneous activities, possession of appropriate devices to manage condition as desired, need of device that reduces burden of managing the condition, any combination thereof, or the like.

A second algorithm may then be used to give predetermined weights to each of the second set of prompts or responses thereto to determine which care pathway(s) is most relevant to the patient. The patient's electronic medical health data, which may include information relating to diagnostic tests, medical examinations, personal trackers, sleep monitors, or any combination thereof, may then be retrieved. The first and/or second set of patient data and the electronic medical health data may then be synthesized and analyzed to determine a holistic treatment recommendation for the patient.

Each prompt/response (or certain ones of the prompts/responses) in the first wave, the second wave, or both the first and second waves may be linked to a mechanism of action. For example, the prompt(s) may be mapped to a behavior that is modifiable to a particular health outcome. This may assist with achieving improved outcomes, e.g., identification of psychological burden, signposting to online resources to reduce distress, proving greater cognitive availability for self-management tasks and improved biomedical and quality of life outcomes, any combination thereof, or the like.

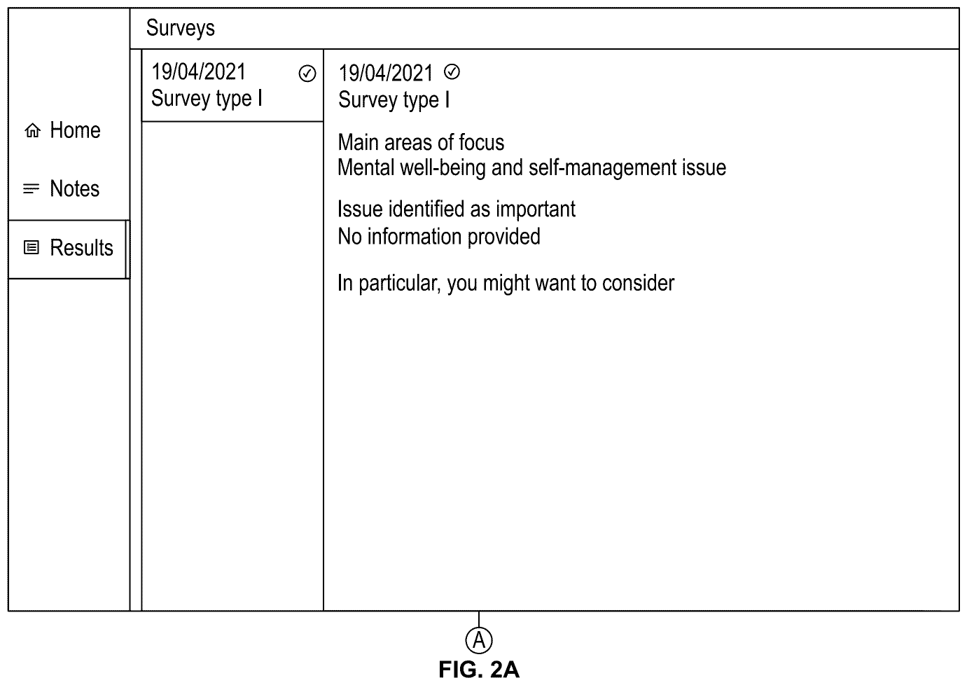

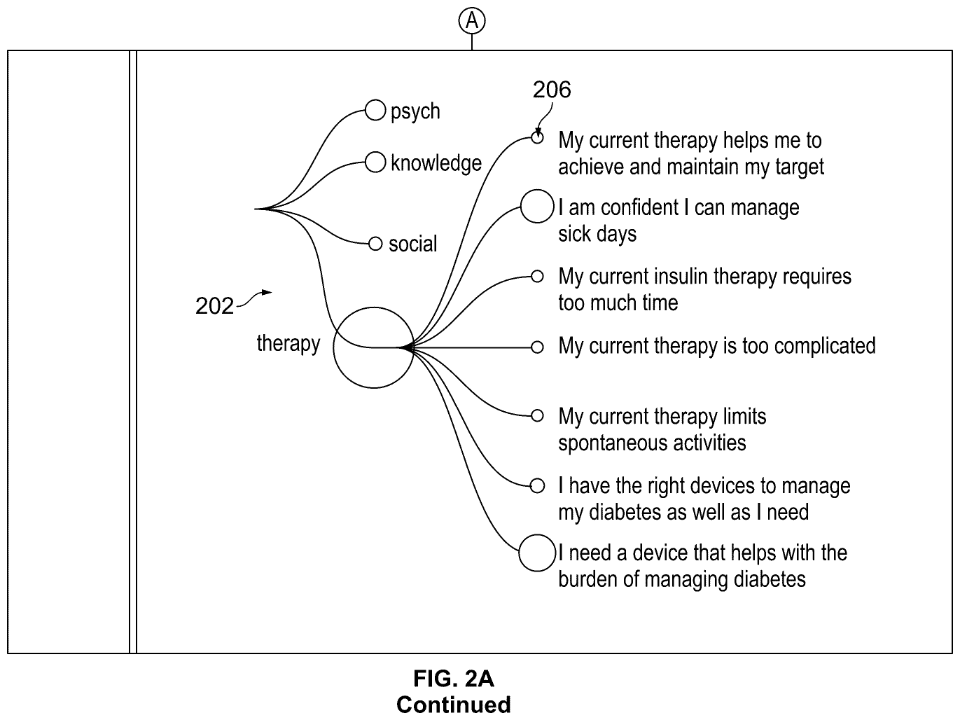

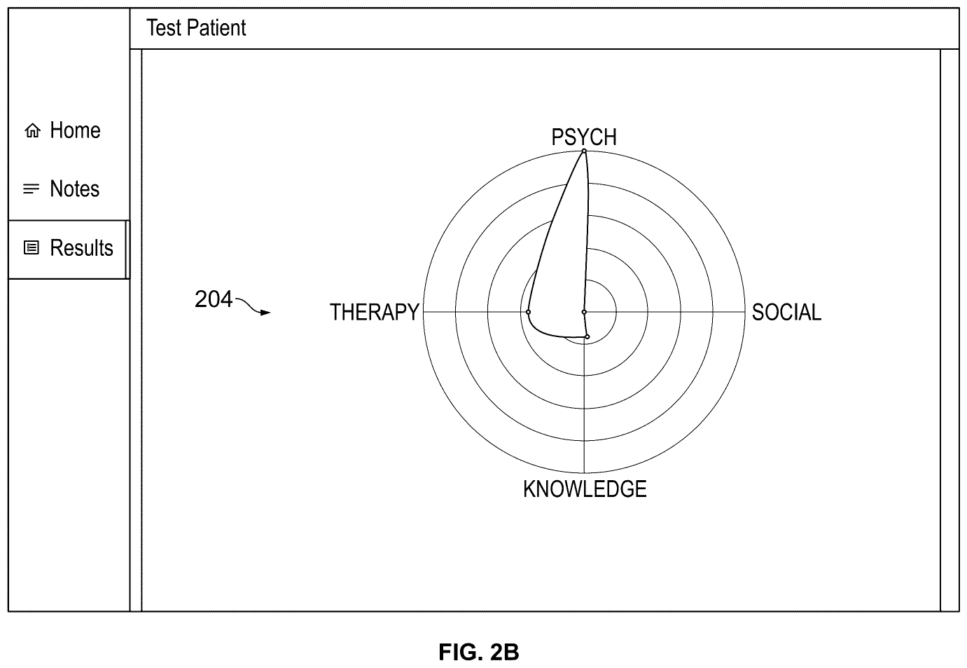

FIGS. 2a, 2b illustrate non-limiting embodiments of a results screen displayed on a display and visible to the patient upon completing the assessment. The results may be used as the basis of discussion between the patient and his/her HCP during the consultation. In the embodiment of FIGS. 2a and 2b, results are presented in three different ways: textually, as a “bubble graph” 202, and as an “overview graph 204. However, it is contemplated that the results may be presented in any suitable manner. On the bubble graph 202, the larger the bubble 206, the greater the determined relevance of the corresponding priority concern area to the patient/participant.

Once the assessment is completed by the patient and submitted electronically, the HCP may review and/or modify the results to ensure alignment with medical results and opinion to assist in arriving at a highly efficient and effective clinical care plan or holistic treatment recommendation based on and incorporating the patient's identified needs.

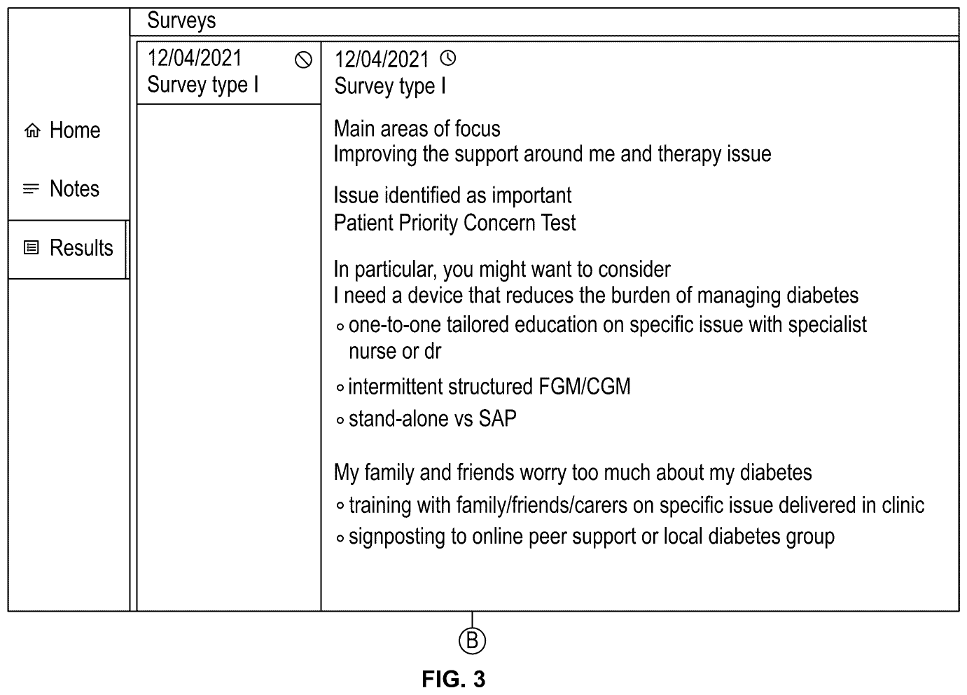

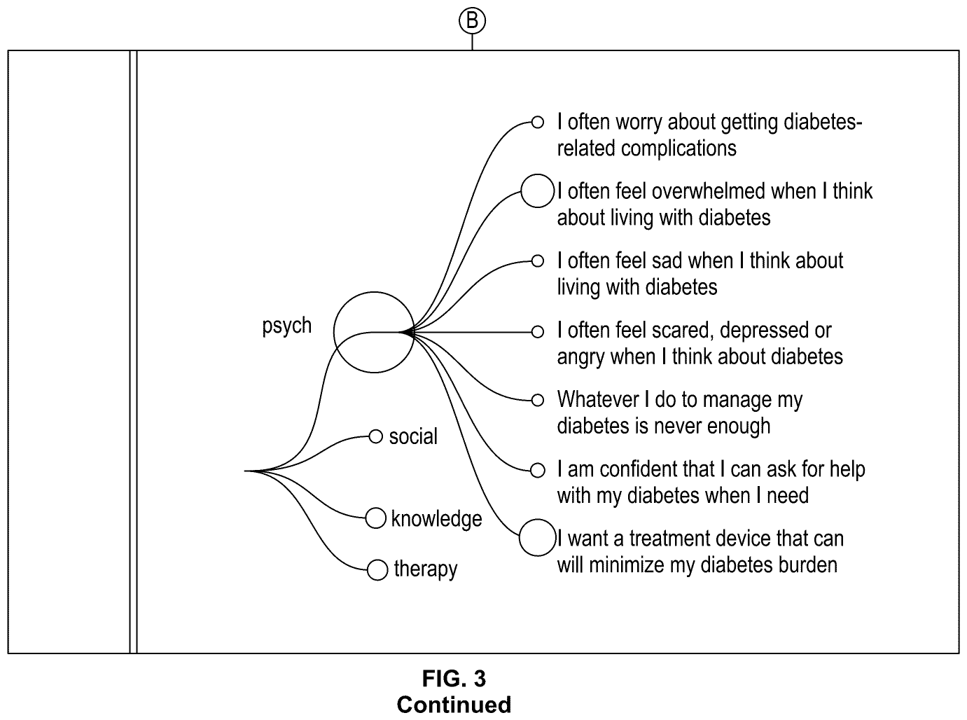

FIG. 3 illustrates a non-limiting embodiment of a results screen displayed on a display and visible to the HCP upon the patient completing the assessment. The results can be viewed from, e.g., the HCP's portal. The HCP may click on the particular patient whose assessment results he/she would like to view to be taken to the results page. In the embodiment of FIG. 3, the results display is similar to the patient-viewable result screen of FIGS. 2a, 2b in that the main priority concerns areas are shown. However, in the HCP view of the embodiment of FIG. 3, bullet point care pathway, high level options for consideration and discussion are also displayed and viewable.

The systems and methods disclosed herein provide an easy-to-navigate digital results page(s) of necessary data to deliver patient-centered care, including identification of facilitators and barriers to behavior change, patient preference data, and patient reported outcome and quality of life insights.

The treatment recommendation for the patient may be displayed on a display associated with the memory device. The treatment recommendation is output in an easy-to-read manner, such as a graphical representation, a figurative representation, written text, audibly, any combination thereof, or the like. In one non-limiting embodiment shown in FIG. 2, the results are presented such that the priority concern areas are positioned at the top of the screen, followed by a biomedical summary, followed by a diagram (e.g., a spider diagram) of how the priority concerns were determined. As briefly discussed above, in the embodiment of FIGS. 2a and 3, the size of bubbles associated with the priority concern areas have been adjusted based on the determined relevance of the particular priority concern area to the patient.

In some embodiments, one or more other diagrams is included to provide an overview of results. For example, the graphical image of FIG. 2b depicts the diversity of patient preferences in how he/she processes information and what he/she finds most impactful and easy to understand/implement. As shown in FIG. 2b, the patient's personal barriers may be mapped onto several axes, namely psychologically-related, social support-related, knowledge-related, and treatment-related to identify pathways for intervention and support. The suggested pathways are intended to clarify the patient's needs and ultimately facilitate a discussion with his/her health care team to develop a plan to address those needs more effectively. The size, order, etc. of the representation (or components thereof) may be associated with the determined relevance of to the patient.

The patient will be able to navigate his/her own personal responses forming the basis for discussion in the clinic visit. The determined priorities may not be overtly related to his/her condition, but will directly impact the patient's self-efficacy, quality of life, and ability to optimize self-management of the condition and should be addressed as such.

In some embodiments, the HCP can make modifications, e.g., influence what information the patient sees and/or how the information is presented to the patient. In some embodiments, the results of multiple assessment taken over a period of time can be viewed and compared.

Although the results of the assessment may be viewed independently by the patient and his/her HCP (e.g., in advance of the routine outpatient consultation), the results may also be viewed by the patient and the HCP together during the consultation and used to form the basis of the discussion to provide patient-centered, goal-focused, actionable care planning, an agreed-upon care plan, and/or a holistic treatment recommendation. The self-management skills and the priority concerns may include high-level suggestions, enabling the doctor to use his/her expertise in deciding which course of action to recommend/prescribe.

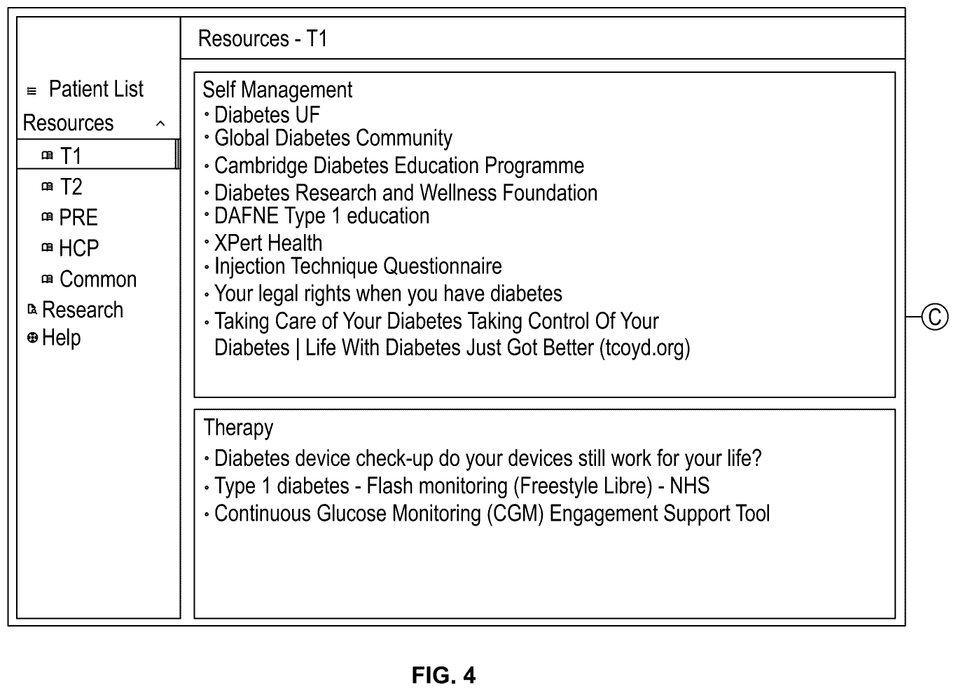

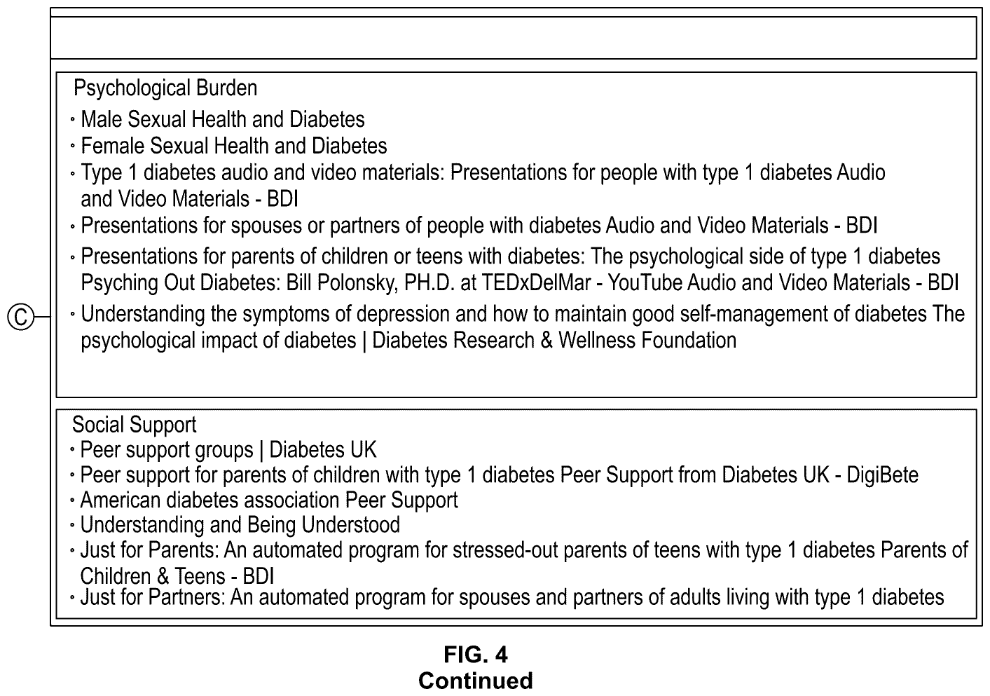

Referring to the non-limiting embodiment of FIG. 4, in some embodiments, the results page is such that the HCP (or the patient) may click on each priority concern area separately and be presented with a choice of one or more best-matched potential care pathway suggestions for each of priority concern areas. The output/results page may include resources associated with the treatment recommendation and/or information associated with using the care treatment recommendation to tailor treatment for a particular condition. For example, the psychological burden and social support priority concern buttons may include more detailed recommendations and signpost resources (e.g., through web links) to where the HCP and/or patient can find specific advice/guidance on the particular problem identified in the results. In cases of diabetes care management, these resources may include, e.g., links to tools on injection technique assessment, social support, weight loss services, or the like. Desirably, the resources are evidence-based, from trusted sources, widely and/or freely available (e.g., on the Internet), or the like. Thus, the HCP is able to have a patient-centered, goal-focused, and action-oriented discussion with the patient. The holistic treatment recommendation may be saved to the patient's electronic health record.

Non-limiting examples of best practice care pathway choices associated with knowledge may include, but are not limited to, one-to-one tailored education on a specific issue (e.g., with dietician), support from certified educator/dietician, signposting to online training resources, peer support signposting to online training, pattern management education, structured education, decision support tools, group structured education, decision support tools, online structured education resources, any combination thereof, or the like. Non-limiting examples of best practice care pathway choices associated with psychological burden may include, but are not limited to, in-house behavioral health specialist support, referral to specialist, behavioral health specialist support, online behavioral health support, any combination thereof, or the like. Non-limiting examples of best practice care pathway choices associated with social support may include, but are not limited to, training with family/friends on specific issue delivered in clinic, signposting to online peer support to local care group, psychosocial support to explore ways of engagement, therapy, one-to-one tailored education on specific issue with specialist, nurse, or doctor, providing resources relating to use of medical devices and/or medical device options, providing choices of therapy/technologies available, providing information regarding group structured education, providing online resources for structured education, providing referrals to midwifery services, providing decision support tools, providing peer support information, any combination thereof, or the like.

Providing such a summary, including care pathway choices and information related thereto may assist in promoting a truly patient-centered discussion, with clarity of the broader, important factors affecting chronic condition self-management. By providing such clarity alongside choices, the patient may make an intrinsically motivated decision, with the support of his/her HCP, about what course of action is most appropriate in the context of his/her lived experience and health needs. Such intrinsically motivated decisions are typically more enduring and powerful than externally imposed instruction, as per self-determination theory.

The e-tools described herein may be embedded with a secure web-based platform accessible to healthcare professionals and patients separately. Current and previous treatment recommendations may be stored such that they may be immediately viewed by the patient and HCP, either independently or collaboratively, via a login. In addition, the patient and/or the HCP may add and save notes to the assessment and/or resulting report, which notes may be private or viewed by the other of the patient or HCP. In some embodiments, the HCP may enter and store notes pertaining to the consultation with the patient in the patient's electronic health record. Fundamentally, the pre-clinic assessment focuses consultation discussions through shared understanding of patient priority concerns and tailoring of care to address those concerns.

In some embodiments, the system may include non-verbal functionality including, but not limited to, audio capacity and/or color functionality replacing text functionality available for use by people with disabilities, low literacy, dyslexia, or the like. In some embodiments, the system may further include inbuilt adaptive features and characteristics for use in immigration status assessment, recruitment processes, and/or social networking (e.g., dating and friendship).

The systems and methods of the embodiments described herein may be adapted for a particular audience, condition, etc. for which they are meant to be used. For example, the tool may be adapted for use by children with chronic health conditions and/or parents of children with chronic health conditions. The system may be further adapted specifically for a particular condition, e.g., diabetes, irritable bowel disease, chronic obstructive pulmonary disorder, cancer, cardiovascular disease, mental health, chronic pain, arthritis, and the like. The system may include features for providing “on demand” psychologist support. The system may be further adapted to provide mood mapping and/or progress perception tools to identify when patients are struggling or thriving, what is happening in other areas of their life that impact these phases, and the like. Artificial intelligence (AI) integration may be used to assist in analyzing trends and patterns from clinical data, highlighting best care pathways for individuals, determined by their results.

Remote consultations and data sharing between health care professionals and their patients through video conferences, telephone, texting, and other eHealth technologies offer opportunities to interact and engage with large number of patients over distance in innovative and cost-effective ways. The embodiments described herein are beneficial because they can be used both in face-to-face interaction and/or in remote interaction. Thus, they are not limited to telemedicine or communication alone. Desirably, the embodiments described herein link crucial data from several different places and bring that data together, reorganizing it into meaningful and understandable forms, including textual (e.g., in multiple languages), audio, color, pictorial, and/or verbal representations—all in one place. These outputs can be accessed by both parties—HCPs and patients—through secure networks either together or individually, to improve outcomes for both parties.

As such, the system and methods described herein may assist in streamlining health, social, and wellness care delivery and/or removing pressure from HCPs to provide “the answer” without having the required information about their patients' individual needs and barriers to effective self-management. Moreover, because the tools match users with treatment options/devices that are sustainable in the long term, they have the potential to create greater economic efficiencies for healthcare systems by improving therapy adherence and minimizing discontinuation of medical device use.

### Example 1

A study was conducted utilizing the systems and methods described herein in the context of diabetic individuals and diabetes care management.

A convenience sampling strategy was adopted based on outpatient patient lists held at each participating center. HCPs approached the principal investigator requesting participation, thus representing a convenience sample.

Forty-nine adults took part, n=31 T1D, (n=18 female); and n=18 T2D (n=10 male, n=4 female, n=4 gender unreported). Three HCPs participated: two general practitioners (GPs) in primary care in the United Kingdom and one endocrinologist in specialist secondary care in the United States. Participant demographics are presented in Table 1.

TABLE 1

| Demographic | | | | |
|---|---|---|---|---|
| characteristic | Type 1 diabetes | | Type 2 diabetes | |
| Age | Mean: | 45.52 | Mean: | 55.43 |
| | (range | 19-67) | (range: | 35-65) |
| Gender | 13 male | (41.9%), | 10 male | (55.6%), |
| | 18 female | (58.1%) | 4 female | (22.2%), |
| | | | 4 not reported | (22.2%) |
| Duration of | Mean: | 21.1 years | Mean: | 14.4 years |
| diabetes | (range | 1-46) | (range: | 3-24) |
| Diagnosed with | 20 no | (64.5%), | 6 no | (33.3%), |
| complications | 11 yes | (35.5%) | 12 yes | (66.7%) |

Participants were asked to complete the pre-clinic assessment, as described in detail above, prior to their routine outpatient visit. HCPs received a brief instruction manual, but no other training was needed or provided. Secure individual links to the questionnaire were sent by HCPs to participants within the seven days prior to the scheduled outpatient appointment. Online completion of the assessment generally took about three to five minutes. As detailed above, the assessment comprised a “smart” adaptive patient survey or questionnaire designed to improve routine outpatient consultations by rapidly identifying patient priorities and presenting them in the context of personalized areas for concern and best-practice care pathways to illuminate consultations. The number of questions presented to participants varied based on their responses to previous questions and priority concerns identified throughout the process. Results were immediately presented to both the participant and the HCP. These results were immediately available and guided the discussion and goal-focused decisions made within the routine clinic appointment.

HCPs were able to use the pre-clinic assessment in the best way that suited their clinical practice in terms of precise timing of when participants completed the assessment. HCPs were provided with lists of support resources for psychological aspects and social aspects of diabetes to signpost to their participants, should those be highlighted as a priority concern area. Therapy and knowledge priority concerns were addressed using usual care pathways at each participating site.

Each of the participants identified two priority concerns. For participants with type 1 diabetes, “Psychological burden of diabetes” was the most common priority concern (T1D n=27, 87.1%), followed by “gaining more skills about particular aspects of diabetes” (T1D n=19, 61.3%), “improving support around me” (n=8, 25.8%), and “diabetes-related treatment issues” (n=8, 25.8%). Burden of diabetes was widespread, as was lack of confidence around self-management. Participants with diabetes-related complications more often prioritized “diabetes related treatment issues” than those without complications. Participants whose latest hemoglobin A1c (HbA1c) was greater than 8.6%were more likely to prioritize “gaining more skills” than those whose A1c was less than 8.5. Men reported greater psychological burden (92.3% vs 83.3%), while women prioritized gaining more skills (66.7% vs 53.9%). Those aged less than 35 years more often prioritized psychological burden than those greater than or equal to 35 years (100% vs 82.6%). Gaining more skills was more frequently a priority concern among those with higher duration of diabetes (44% among those having diabetes for less than 10 years vs 68% among those having diabetes for more than 10 years).

Similarly, psychological burden of diabetes was the primary concern for participants with T2D (n=18,100%), followed by “gaining more skills about aspects of diabetes” (n=7, 38.9%), “improving support around me” (n=7, 38.9%), and “diabetes-related treatment issues” (n=4; 22.2%). Feeling sad about living with diabetes and frustrated that efforts are never enough (each n=15, 83.3%), feeling scared (n=11, 61.1%), and lacking confidence (n=9, 50%), were all commonly reported. Participants who had diabetes for more than 10 years were more likely to report lack of social support as a priority concern (50% vs 25%); those with diabetes duration less than 10 years were more likely to report gaining more skills as a priority concern (50% vs 20%).

Table 2 presents data broken down by duration of diabetes.

TABLE 2

| | Type 1 diabetes (T1D) | | | | Type 2 diabetes (T12) | | |
|---|---|---|---|---|---|---|---|
| | 1-10 | 11-20 | 21-30 | 31+ | 0-10 | 11-20 | 21-30 |
| | years | years | years | years | years | years | years |
| | (n = 9) | (n = 9) | (n = 4) | (n = 9) | (n = 4) | (n = 9) | (n = 4) |
| The psychological | 9 | 9 | 3 | 6 | 4 | 7 | 3 |
| burden of diabetes | (100%) | (100%) | (75%) | (66.67%) | (100%) | (100%) | (100%) |
| Gaining more | 4 | 5 | 3 | 7 | 2 | 1 | 1 |
| skills or | (44.44%) | (55.56%) | (75%) | (77.78%) | (50%) | (14.29%) | (33.33%) |
| information about | | | | | | | |
| particular aspects | | | | | | | |
| of diabetes | | | | | | | |
| Improving the | 3 | 2 | 1 | 2 | 1 | 4 | 1 |
| support around me | (33.33%) | (22.22%) | (25%) | (22.22%) | (25%) | (57.14%) | (33.33%) |
| Diabetes-related | 2 | 2 | 1 | 3 | 1 | 2 | 1 |
| treatment issue | (22.22%) | (22.22%) | (25%) | (33.33%) | (25%) | (28.57%) | (33.33%) |
| (insulin) or | | | | | | | |
| glucose | | | | | | | |
| monitoring) | | | | | | | |

Table 3 presents data broken down by age and gender.

TABLE 3

| | Type 1 diabetes (T1D) | | | | | Type 2 diabetes (T12) | | | |
|---|---|---|---|---|---|---|---|---|---|
| | <35 | 35-50 | 51+ | | | 35-50 | 51+ | | |
| | (n = 8) | (n = 8) | (n = 15) | F | M | (n = 2) | (n = 12) | F | M |
| The psychological | 8 | 7 | 12 | 15 | 12 | 2 | 12 | 4 | 10 |
| burden of diabetes | | | | | | | | | |
| Gaining more skills or | 4 | 5 | 10 | 12 | 7 | 2 | 2 | 1 | 3 |
| information about | | | | | | | | | |
| particular aspects of | | | | | | | | | |
| diabetes | | | | | | | | | |
| Improving the support | 2 | 1 | 5 | 4 | 4 | 0 | 6 | 2 | 4 |
| around me | | | | | | | | | |
| Diabetes-related | 2 | 3 | 3 | 3 | 3 | 0 | 4 | 1 | 3 |
| treatment issue (insulin) | | | | | | | | | |
| or glucose monitoring) | | | | | | | | | |

Tables 4 and 5 present data broken down by HbA1c.

TABLE 4

| | | | | | Type 2 diabetes (T12) | | | | | |
|---|---|---|---|---|---|---|---|---|---|---|
| | Type 1 diabetes (T1D) | | | | | | | | ″I ″I don't | |
| | <8.5% | | >8.6% | | <8.5% | | >8.6% | | know″ | |
| | (n = 19) | | (n = 12) | | (n = 3) | | (n = 9) | | (n = 6) | |
| The psychological | 16 | (84.21%) | 11 | (91.67%) | 3 | (100%) | 9 | (100%) | 6 | (100%) |
| burden of diabetes | | | | | | | | | | |
| Gaining more skills or | 8 | (42.11%) | 11 | (91.67%) | 2 | (66.67%) | 5 | (55.56%) | 0 | (0%) |
| information about | | | | | | | | | | |
| particular aspects of | | | | | | | | | | |
| diabetes | | | | | | | | | | |
| Improving the support | 6 | (31.58%) | 2 | (16.67%) | 1 | (33.33%) | 3 | (33.33%) | 3 | (50%) |
| around me | | | | | | | | | | |
| Diabetes-related | 8 | (42.11%) | 0 | (0%) | 0 | (0%) | 1 | (11.1%) | 3 | (50%) |

treatment issue
(insulin) or glucose
monitoring)

TABLE 5

| Self- | Type 1 diabetes | | Type 2 diabetes | |
|---|---|---|---|---|
| Reported A1c | (T1D; n = 31) | | (T2D; n = 14) | |
| Underestimated | 6 | (19.4%) | 2 | (14.3%) |
| Overestimated | 7 | (22.6%) | 2 | (14.3%) |
| Correct | 18 | (58.1%) | 4 | (28.6%) |
| Don't know | 0 | (0%) | 6 | (42.9%) |

As part of the study, participants were asked to provide their latest HbA1c result. During the study, it was noticed that the participant's subjective recall differed somewhat from the latest recorded HbA1c on the participants' medical records. As such, both subjective and objective HbA1c data, which were available for 45 participants (n=31 with T1D; n=14 with T2D), underwent comparative analysis. While P values are not provided due to small participant numbers, results provided an understanding of the level of accuracy surrounding subjective recall of individual HbA1c results. Overall, accurate recollection of HbA1c was poor, with only 22 participants (48.9%) having correct recall (Table 5). Six participants (13.3%) did not know their A1c, eight (17.8%) reported it as lower than it actually was, and nine (20.0%) reported it as higher than it actually was. Individuals with T1D were more likely to correctly report their A1c (n=18, 58.1%) than those with T2D (n=4, 28.6%); overestimation was reported by n=7 with T1D (22.6%) versus n=2 with T2D (14.3%); underestimation was reported by n=6 with T1D (19.4%) and n=2 with T2D (14.3%). People with T1D who overestimated their A1c were more likely to have “gaining more skills” (n=6; 85.7%) as a priority concern. People with T1D correctly recalling their A1c were more likely to have “psychological burden” as a priority concern (n=17; 94.4%). Those who did not know their A1c (all with T2D) reported “psychological burden” as a priority concern.

The systems and methods of the embodiments presented herein were acceptable to all participants based on participant and HCP feedback, and were feasible for delivery in routine care. High levels of diabetes psychological burden were identified amongst participants. In addition, a need to gain more skills about particular aspects of diabetes management was prioritized by the majority of participants.

As highlighted by one participating clinician, the time pressure of approximately 15 minutes per visit in routine consultations is a barrier to addressing psychological aspects in management of individuals with T1D and T2D. Using the intervention tool, with signposting to psychological and/or social support enabled these areas of diabetes self-management to be discussed within the consultation, whereas this would not usually be possible. There may also be a conscious bias to avoid such discussions, as HCPs often feel ill-equipped to provide solutions.

Addressing the psychosocial challenges of T1D and T2D was shown to be effective. It is well known that factors including (but not limited to) affordability, treatment complexity, potential medication side effects, and poor health literacy all play a major role in patients' ability to follow recommended treatment plans. Furthermore, the impact of psychological factors (e.g., diabetes distress) must be considered for optimal biomedical and quality of life outcomes. Novel, holistic, patient-centered, individualized care deliverable in routine clinic settings is required to help people with diabetes explore and better understand their barriers and facilitators, using intrinsic motivation to achieve better personal outcomes. This is in direct contrast to externally imposed targets that currently exist. Such care, harboring the power of intrinsic motivation, will achieve better personal outcomes in comparison to externally imposed targets by HCPs, which are often felt judgmental and unattainable.

The study of Example 1 demonstrated that using patient-centered e-tools within routine care can improve the healthcare experience for both HCPs and their patients. The e-tool system and methods described herein enable ascertaining the patient's agenda in a time efficient manner, adding value for the individual attending the consultation. In fact, the consultation length may actually be reduced. As such, all participating HCPs reported that they would recommend the tool to their colleagues.

The tools described herein have been found to be acceptable and feasible for use in routine care. Gaining more skills and addressing the psychological burden and other priority concern areas of chronic conditions are high-priority areas that must be addressed to reduce high levels of distress. Gaining more skills and addressing priority concern areas related to chronic conditions are high priority areas that must be addressed to reduce high levels of distress.

The systems and methods described herein provide dynamic adaptive approaches to identifying and addressing individuals' needs at their clinical encounters and throughout each stage of their life. Importantly, the patient is at the heart of decision-making with the availability of therapy, education, and support tailored to provide individualized care. The novel systems and methods support HCPs in providing care pathways that are mapped to individuals' priorities by unique linkages between electronic health records, personal computing technologies, and core assessment.

It is contemplated that the patient may also access and review his/her assessment results/information outside of a routine health consultation. For example, the systems and methods described herein may be offered to a patient periodically or at any predetermined time, and the patient may have the option to contact his/her HCP only when and if the patient has questions or desires feedback.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be understood that the term “computer,” as used herein, is intended to refer to any machine including at least a processor therein. For example, “computer” may refer to a desktop computer, laptop computer, smart phone, tablet, or the like.

It should be noted that the terms “exemplary” and “example” as used herein to describe various embodiments are intended to indicate that such embodiments are possible examples, representations, and/or illustrations of possible embodiments (and such terms are not intended to connote that such embodiments are necessarily extraordinary or superlative examples).

Although only a few embodiments have been described in detail in this disclosure, those skilled in the art who review this disclosure will readily appreciate that many modifications are possible without materially departing from the novel teachings and advantages of the subject matter described herein. The order or sequence of any process or method steps may be varied or re-sequenced according to alternative embodiments. Other substitutions, modifications, changes, and omissions may also be made in the design, operating conditions, and arrangement of the various exemplary embodiments without departing from the scope of the present invention.

1. A computer-implemented method of formulating and implementing a holistic treatment recommendation for diabetes management, the method performed by one or more processors, the method comprising:
retrieving a patient's electronic medical health data;
receiving a first set of patient data from a patient database stored in a memory device, the first set of patient data being based on responses to a first set of prompts relating to a plurality of priority concern areas associated with diabetes self-care management;
determining, using a processor giving predetermined weights to each of the first set of prompts or responses thereto, which of the priority concern areas are relevant to the patient;
receiving a second set of patient data from the patient database stored in the memory device, the second set of patient data being based on responses to a second set of prompts relating to the one or more of the priority concern areas determined to be relevant to the patient;
determining, using a processor giving predetermined weights to each of the second set of prompts or responses thereto, a first priority concern area and a second priority concern area, the first priority concern area representing a most relevant of the plurality of priority concern areas, the second priority concern area representing a second most relevant of the plurality of priority concern areas;
determining, based on the first and second priority concern areas and the electronic medical health data, a holistic treatment recommendation for diabetes management;
retrieving, from the memory device, a plurality of external links associated with the holistic treatment recommendation, the plurality of external links having a first set of external links and a second set of external links, the first set of external links being associated with the first priority concern area, the second set of external links being associated with the second priority concern area;
displaying, on a first display device, a first icon and a second icon, the first icon being representative of the first priority concern area, the second icon being representative of the second priority concern area;
responsive to a selection of the first icon, displaying, on the first display device, a first plurality of concerns associated with the first priority concern area, each of the first plurality of concerns being associated with one or more external links from the first set of external links;
responsive to a selection of the second icon, displaying, on the first display device, a second plurality of concerns associated with the second priority concern area, each of the second plurality of concerns being associated with one or more external links from the second set of external links; and
implementing the holistic treatment recommendation for diabetes management for at least one of the first priority concern area and the second priority concern area.

2. The method of claim 1, wherein the priority concern areas include psychological burden, therapy aspects, social support, knowledge, or any combination thereof.

3. The method of claim 2, wherein the first set of prompts includes prompts relating to psychological burden, the prompts being further related to the patient's feelings, beliefs, motivations, or any combination thereof.

4. The method of claim 2, wherein the first set of prompts includes prompts relating to therapy aspects, the prompts being further related to treatment regimens, medication side effects, use of medical devices, education, or any combination thereof.

5. The method of claim 2, wherein the first set of prompts includes prompts relating to social support, the prompts being further related to resources, environment, or any combination thereof.

6. The method of claim 2, wherein the first set of prompts includes prompts relating to knowledge, the prompts being further related to use of medical devices, treatment regimens, lifestyle, or any combination thereof.

7. The method of claim 1, wherein the patient's electronic medical health data includes information relating to diagnostic tests, medical examinations, personal trackers, sleep monitors, or any combination thereof.

8. The method of claim 1, wherein the holistic treatment recommendation includes care pathways and/or resources associated with the first priority concern area and the second priority concern area.

9. The method of claim 1, further comprising saving the holistic treatment recommendation to the patient's electronic health record.

10. The method of claim 1, wherein at least one of the first or second set of prompts is a statement, and the response thereto indicates how strongly the patient agrees with the statement.

11. The method of claim 10, wherein the predetermined weight given to a strong agreement or strong disagreement with the statement is greater than the predetermined weight given for mild agreement or mild disagreement with the statement.

12. The system of claim 1, wherein the predetermined weights correspond with prompts or responses thereto determined to be relevant to one or more of the plurality of priority concern areas.

13. A computing system comprising:
one or more computers and one or more storage devices storing instructions that are operable, when executed by the one or more computers, to cause the one or more computers to perform operations comprising:
retrieving a patient's electronic medical health data;
receiving a first set of patient data from a patient database stored in a memory device, the first set of patient data being based on responses to a first set of prompts relating to a plurality of priority concern areas associated with diabetes self-care management;
determining, using a processor giving predetermined weights to each of the first set of prompts or responses thereto, which of the priority concern areas are relevant to the patient;
receiving a second set of patient data from the patient database stored in the memory device, the second set of patient data being based on responses to a second set of prompts relating to the one or more of the priority concern areas determined to be relevant to the patient;
determining, using a processor giving predetermined weights to each of the second set of prompts or responses thereto, a first priority concern area and a second priority concern area, the first priority concern area representing a most relevant of the plurality of priority concern areas, the second priority concern area representing a second most relevant of the plurality of priority concern areas;
determining, based on the first and second priority concern areas and the electronic medical health data, a holistic treatment recommendation for diabetes management;
retrieving, from the memory device, a plurality of external links associated with the holistic treatment recommendation, the plurality of external links having a first set of external links and a second set of external links, the first set of external links being associated with the first priority concern area, the second set of external links being associated with the second priority concern area;
displaying, on a first display device, a first icon and a second icon, the first icon being representative of the first priority concern area, the second icon being representative of the second priority concern area;
responsive to a selection of the first icon, displaying, on the first display device, a first plurality of concerns associated with the first priority concern area, each of the first plurality of concerns being associated with one or more external links from the first set of external links;
responsive to a selection of the second icon, displaying, on the first display device, a second plurality of concerns associated with the second priority concern area, each of the second plurality of concerns being associated with one or more external links from the second set of external links;
displaying, on a second display device, the first icon and the second icon;
implementing the holistic treatment recommendation for diabetes management for at least one of the first priority concern area and the second priority concern area.

14. The method of claim 1, further comprising:
displaying, on a second display device, the first icon and the second icon; and
responsive to a selection of the first icon, displaying, on the second display device, the first plurality of concerns, each of the first plurality of concerns being associated with one or more external links from a subset of the first set of external links.

15. The method of claim 14, further comprising: responsive to a selection of the second icon, displaying, on the second display device, the second plurality of concerns, each of the second plurality of concerns being associated with one or more external links from a subset of the second set of external links.

16. The method of claim 15, wherein the subset of the first set of external links and the subset of the second set of external links represent health-care provider approved external links.

17. The method of claim 1, further comprising transmitting, via a communication device, the subset of the first set of external links and the subset of the second set of external links.

18. The method of claim 1, wherein the first set of prompts is based on the patient's electronic medical health data.

19. The method of claim 18, wherein the second set of prompts is based on the patient's electronic medical health data.

20. The method of claim 1, wherein a size of the first icon and a size of the second icon is dependent on the relevance of the priority concern area to the patient, wherein the largest icon is the icon associated with the first priority concern area.
